(19) United States
(12) Patent Application Publication
(10) Pub. No.: US 2011/0262489 A1
(43) Pub. Date: Oct. 27, 2011

(54) HYALURONIC ACID CRYOGEL - COMPOSITIONS, USES, PROCESSES FOR MANUFACTURING

(76) Inventor: Xiaobin Zhao, Cambridge (GB)

(21) Appl. No.: 13/063,280

(22) PCT Filed: Sep. 9, 2009
(86) PCT No.: PCT/GB2009/051140

§ 371 Date: Jul. 19, 2011

(30) Foreign Application Priority Data
Sep. 10, 2008 (GB) 0816496.4

(51) Int. Cl. A61K 31/738; A61Q 19/08; A61P 17/02; A61K 9/00; A61K 8/73

(52) U.S. Cl. 424/400; 514/54

(57) ABSTRACT

There is provided a process for preparing a HA cryogel, the process comprising the steps of combining HA, a cross-linking agent and a solvent to form a solution, freezing the solution before the formation of less than 10% of the cross-linking bonds of the cross-linked HA cryogel formed and thawing the solution. An HA cryogel is also provided, in particular an HA cryogel obtained from this process.

### FIELD OF INVENTION

The present invention relates to a hyaluronic acid cryogel, its preparation and its use. More particularly, the invention relates to a hyaluronic acid cryogel as an implant in the field of biomedical engineering, the preparation of such a cryogel, and the use of said cryogel.

### BACKGROUND OF THE INVENTION

Hyaluronic acid (also known as hyaluronan or sodium hyaluronate, and abbreviated to as HA) is a non-sulfated glycosaminoglycan which is distributed widely throughout connective, epithelial, and neural tissues. It is one of the main components of the extracellular matrix, contributes significantly to cell proliferation and migration, and may also be involved in the progression of some malignant tumors.

For example, HA is a major component of the synovial fluid and has been found to increase the viscosity of the fluid. Along with lubricin, it is one of the fluid's main lubricating components. HA is an important component of articular cartilage, where it is present as a coat around each cell (chondrocyte). When aggrecan monomers bind to HA in the presence of link protein, large highly negatively charged aggregates form. These aggregates imbibe water and are responsible for the resilience of cartilage (its resistance to compression). The molecular weight (size) of HA in cartilage decreases with age, but the amount increases.

HA is also a major component of skin, where it is involved in tissue repair. When skin is excessively exposed to UVB rays, HA acts as a free radical scavenger, absorbing free radicals to degrade. The skin becomes inflamed (sunburn), the cells in the dermis stop producing as much HA and increase the rate of its degradation. HA degradation products also accumulate in the skin after UV exposure.

While it is abundant in extracellular matrices, HA also contributes to tissue hydrodynamics, movement and proliferation of cells, and participates in a number of cell surface receptor interactions, notably those including its primary receptor, CD44. Upregulation of CD44 is widely accepted as a marker of cell activation in lymphocytes. HA's contribution to tumor growth may be due to its interaction with CD44. CD44 participates in cell adhesion interactions required by tumor cells. Although HA binds to CD44, there is evidence to support that HA degradation products transduce their inflammatory signal through Toll-like receptor 2 (TLR2), TLR4, or both TLR2 and TLR4, in macrophages and dendritic cells. TLR and HA play a role in innate immunity.

The structure of HA is well characterized. It is composed of repeated units of disaccharide of D-glucuronic acid and D-N-acetylglucosamine, linked together via alternating β-1,4 and β-1,3 glycosidic bonds. HA can be 25,000 disaccharide repeats in length. The molecular weight of HA can range from 5,000 to 20,000,000 Da in vivo. The average molecular weight in human synovial fluid is 3-4 million and HA purified from human umbilical cord is 3,140,000 Da.

HA is synthesized by a class of integral membrane proteins called HA synthases, of which vertebrates have three types: HAS1, HAS2, and HAS3. These enzymes lengthen HA by repeatedly adding glucuronic acid and N-acetylglucosamine to the nascent polysaccharide as it is extruded through the cell membrane into the extracellular space. HA can be commercially produced from animal sources. For example, HA can be produced from rooster combs, umbilical cords, and cartilage of joints or produced by fermentation. The average molecular weight of HA varies according to sources and generally falls within the range 60,000 to 14,000,000 Da.

HA is nontoxic, non-immunogenic and biodegradable. HA is degraded by a family of enzymes called hyaluronidases. In humans, there are at least seven types of hyaluronidase-like enzymes, several of which are tumor suppressors. The degradation products of HA, the oligosaccharides and very low molecular weight HA, exhibit pro-angiogenic properties. In addition, recent studies showed that HA fragments, not native high molecular mass of HA, can induce inflammatory responses in macrophages and dendritic cells in tissue injury and in skin transplant rejection.

HA has been widely accepted for biomedical applications. For example, the first HA biomedical product, Healon, was developed in the 1970s by Dr EA Balazs and during 1980s and it is approved for use in eye surgery (i.e., corneal transplantation, cataract surgery, glaucoma surgery and surgery to repair retinal detachment).

HA is also used to treat osteoarthritis of the knee. Such treatments, called viscosupplementation, are administered as a course of injections into the knee joint and are believed to supplement the viscosity of the joint fluid thereby lubricating the joint, cushioning the joint and producing an analgesic effect. It has also been suggested that HA has positive biochemical effects on cartilage cells. However, some placebo controlled studies have cast doubt on the efficacy of HA injections, and HA is recommended primarily as a last alternative to surgery.

Due to its high biocompatibility and its common presence in the extracellular matrix of tissues, HA is gaining popularity as a biomaterial scaffold in tissue engineering research.

HA may also be used postoperatively to induce tissue healing and can be used to promote the ulcer hearing rapidly.

In the tissue engineering field, scaffold is essential to provide a three-dimensional porous structure to support tissue formation. Scaffolds usually serve at least one of the following purposes: enabling cell attachment and migration; delivery and retention of cells and biochemical factors; enabling diffusion of vital cell nutrients and expressed products; exertion of certain mechanical and biological influences to modify the behaviour of the cell phase.

In the search for an ideal scaffold, many different materials (natural and synthetic, biodegradable and permanent) have been investigated. Examples of these materials are collagen, or some linear aliphatic polyesters.

A commonly used synthetic material is PLA—polylactic acid. This is a polyester which degrades within the human body to form lactic acid; a naturally occurring chemical which is easily removed from the body. Similar materials are polyglycolic acid (PGA) and polycaprolactone (PCL). Their degradation mechanism is similar to that of PLA, but they exhibit respectively a faster and a slower rate of degradation compared to PLA.

Scaffolds may also be constructed from natural materials. In particular different derivatives of the extracellular matrix have been studied to evaluate their ability to support cell growth. Proteins, such as collagen or fibrin, and polysaccharidic materials, like chitosan or glycosaminoglycans (GAGs), have all proved suitable in terms of cell compatibility, but some issues with potential immunogenicity still remain. Among GAGs, HA, is one of the possible choices as scaffold material.

To tailor the biodegradation rate of HA based scaffold for tissue engineering, chemical modification is essential. It includes derivatisation and crosslinking to tailor the biodegradation rate and the mechanical properties.

As tissue fillers, HA derivatives represent an alternative treatment option for the aging face, particularly for facial lines, for lip augmentation and for treatment of distensible atrophic facial scarring. Since 2003 the FDA has approved HA injections for filling soft tissue defects such as facial wrinkles. These tissue fillers are analogous to collagen injections but have the advantages of longer lasting effects and decreased risk of allergic reaction. Among the tissue fillers, Restylane, Hylaform, Juvederm, Belotero, Puragen and many others are the commercially available HA derivatives marketed in Europe while Restylane and Hylaform are also FDA approved. These fillers contain naturally occurring HA molecules that are chemically cross-linked in order to increase the duration in vivo. Restylane family including Restylane, Perlane, and Restylane Fine Lines (Q-Med, Uppsala, Sweden; Medicis, Arizona, USA) are constituted of Nonanimal Stabilized Hyaluronic Acid (NASHA), which is derived from a process using 1,4-butanediol diglycidyl ether (BDDE) as a crosslinking agent to form ether cross-links between the two hydroxyl groups of HA molecules. Hylaform family including Hylaform Fine lines, Hylaform Plus and Hylaform (Inamed Corporation, California, USA) is made of Hylan B gel. Hylan B gel is derived from a crosslinking process using divinyl sulfone (DVS) in which the crosslinking is also through the hydroxyl groups of HA to form sulfonyl-bis-ethyl-crosslinks between HA molecules. Juvederm family including Juvederm 18, Juvederm 24, Juvederm 24HV and Juvederm 30 (Leaderm, France) is available in HA concentrations of 18 mg/ml (Juvederm 18) or 24 mg/ml (Juvederm24, 24HV and 30). They are crosslinked HA products using BDDE as the crosslinking agent like Restylane, but it claims to be in a homogeneous gel form rather than in particle forms. Recently, there are two new HA based dermal fillers launched in Europe and both are claimed to use double cross-linking technology, i.e. Puragen (Mentor Corporation, California) and Belotero/Esthelis (Anteis, Switzerland). Puragen is based on a DXL technology in which HA is crosslinked via both ether-crosslinkage and ester-crosslinkage, while Belotero is based on a technology named as CPM (Cohesive Polydensified Matrix) in which BDDE is used as a crosslinking agent. After the primary crosslinking with the ether-bond formation, low molecular weight (LMW) HA is introduced into the matrix for secondary crosslinking among HA molecules.

It is known that HA should be a good base for a soft tissue augmentation material but each HA based dermal filler has its own distinct characteristics with variations in physico-chemical properties such as rheological properties, gel particle sizes and durability. Understanding the differences among tissue fillers will enable physicians to choose the appropriate commercial products for each individual patient.

In the prior art, chemical modification of HA takes place at a temperature of above 0° C., and typically is ranged from 2 to 60° C. In order to promote the reaction, most of the reactions are carried out at room temperature to 60° C. Some of the reactions, when other biological material is involved, are carried out at a temperature controlled to be between 2 to 8° C. After chemical modification, the material can be freeze dried to form a porous structured material. However, due to the nature of the softness of the material, it is difficult to recover the pore structure after hydrogel formation.

It is already well known that HA is not thermo-stable and easy to degrade at acidic or alkaline conditions at high temperature; conditions which are commonly used for chemical modification of HA. This will significantly change the yield of production of HA derivatives, and the biocompatibility of the finished products, due to the generation of HA degradation fragments. In order to compensate for the degradation, more reactants such as crosslinking agents are required. This helps to keep the reaction time short, and thus helps to avoid degradation. However, it is difficult (due to the high molecule weight of HA) to achieve successful crosslinking to create a well organized crosslinked network. Additionally, further purification to remove the toxic reactant residue is also problematic.

Therefore, there is a long felt need for an ideal chemical modification method for the modification of hyaluronic acid, and to produce an ideal biodegradable scaffold for tissue engineering to overcome the above problems.

Cryogels are a new class of materials with a highly porous structure and having a broad variety of morphologies. Cryogels are produced using a cryotropic gelation technique.

Cryotropic gelation (cryogelation or cryostructuration) is a specific type of gel-formation which takes place as a result of cryogenic treatment of the systems potentially capable of gelation. [Lozinsky, V. I., Vainerman, E. S., Rogozhin, S. V., Method for the preparation of macroporous polymer materials, SU Inventor's Certificate No. 1008214 (1982).] The essential feature of cryogelation is compulsory crystallization of the solvent such as water. This distinguishes cryogelation from chilling-induced gelation, when the gelation takes place on decreasing temperature.

A typical feature of cryogelation is the ability to produce a system of interconnected macropores. The macropore size can be as large as a few hundred microns. The cryogels often have sponge-like morphology, which is contrary to traditional continuous monophase gels that are produced from the same precursors, but at temperatures above freezing.

Cryogels are mechanically strong which is sometimes desirable in certain applications such as wound dressing films and foams.

The production of cryogels in general is well documented (Vide e.g. Kaetsu, I., Adv. Polym. Sci. 105: 81 (1993); Lozinsky, V. I. and Plieva, F. M., Enzyme Microb. Tech-No I. 23: 227-242 (1998); and Hassan, Ch. M. and Peppas, N. A., Adv. Polym. Sci. 151:37 (2000).

Poly (vinyl alcohol) (PVA) cryogel are the most widely studied due to their easy availability. When cooling an aqueous solution of PVA to a temperature of below 0° C. the ratio between gelling of the PVA and the crystallization of water is such that cryogels are easily formed. The repeated freeze/thawing cycle will create a water-insoluble PVA gel, which can be further chemically modified if necessary.

The preparation of cryogels by polymerizing an aqueous solution of acrylamide and N,N′-methylene-bis-acrylamide in the presence of a radical polymerization initiator under chilling to a temperature below 0° C. is disclosed, e.g. by E. M. Belavtseva et al., Colloid & Polymer Sci. 262: 775-779 (1984); V. I. Lozinsky et al., Colloid & Polymer Sci. 262: 769-774 (1984) and D. G. Gusev et al., Eur. Polym. J. Vol. 29, No. 1, 49-55, 1993. Further cryogels prepared by polymerizing an aqueous solution of monomers under chilling at a temperature at which solvent in the system is partially frozen with the dissolved substances concentrated in the non-frozen fraction of the solvent is disclosed in SU Inventor's Certificate No. 1008214.

Vladimir I. Lozinsky, et al published a paper (Trends in Biotechnology Volume 21, Issue 10), October 2003, Pages 445-451) to give an introduction about the mechanism and application of cryogel for immobilisation of biomolecules and cells, and for chromatographic bio-separation application of molecules.

H. Kirsebom published a paper titled as macroporous scaffolds based on chitosan and bioactive molecules (Journal of Bioactive and Compatible Polymers, Vol. 22, No. 6, 621-636 (2007)). In this paper chitosan-based macroporous scaffolds for tissue engineering applications are developed by cryogelation in aqueous media. The cryogels obtained are modified using a new RGD-containing peptide developed in this laboratory. A RGD-containing peptide is chemically attached to the surface of the cryogels to improve cell adhesion to the 3D-structure chitosan-based scaffolds. The synthesis, physico-chemical, and biological evaluations of the system are described, and the optimization of the formulations is carried out by varying the reaction parameters. Fibroblasts and endothelial cells are used in cell cultures to determine cell behavior and the cytocompatibility of the macroporous cryogels. Cell spreading and actin cytoskeleton organization process are assessed by confocal microscopy. Cells colonize the porous structure of the chitosan-based cryogel and are observed to be growing inside the pores.

AK Bajpai, Rajesh Saini disclosed a paper for preparation and characterization of biocompatible spongy cryogels of poly(vinyl alcohol)-gelatin and study of water sorption behaviour (Polymer International Volume 54 Issue 9, Pages 1233-1242, 2007). In this paper, porous biocompatible spongy hydrogels of poly(vinyl alcohol) (PVA)-gelatin were prepared by the freezing-thawing method and characterized by infrared and differential scanning calorimetry. The ‘cryogels’ were evaluated for their water-uptake potential and the influence of various factors, such as the chemical architecture of the spongy hydrogels, pH and the temperature of the swelling bath, on the degree of water sorption by the cryogels was investigated. The biocompatibility of the prepared materials was assessed by in vitro methods of blood-clot formation, platelet adhesion, and percent haemolysis. It was noticed that with increasing concentration of PVA and gelatin the biocompatibility increased, while a reduced biocompatibility was noted with an increasing number of freeze-thaw cycles.

Martin Hedström, Fatima Plieva, Igor Yu. Galaev and Bo Mattiasson (Anal and Bioanal Chem. 2008, 390(3) 907-912) disclosed a method to prepare a novel monolithic macroporous material by cross-linking hen egg albumin (HEA) and chitosan with glutaraldehyde at subzero temperatures. A macroporous cryogel structure allowed efficient mass transport of solutes within the material. In one application, albumin was partially replaced with active enzymes (glucose oxidase and horseradish peroxidase) resulting in the production of macroporous biocatalyst preparations suitable for flow-injection analysis of glucose in the low millimolar range. In another application, the proteolytic enzymes savinase and esperase were coupled to the macroporous structure via free amino groups on the pore walls using glutaraldehyde as cross-linker/spacer agent. The low hydraulic resistance of the matrix allowed for the development of a generic, high-performance online protein digestion system utilizing the wall-bound proteases.

Judith A. Piermaria, Mariano L. de la Canal and Analía G. Abraham disclosed a study on gelling properties of kefiran, a food-grade polysaccharide obtained from kefir grain under cryogenic condition (Food Hydrocolloids Volume 22, Issue 8, December 2008, Pages 1520-1527). The storage modulus (G′) in cryogels was 35 times higher than the value obtained for the solution.

By reviewing the prior art, the cryogels are mainly based on PVA, a synthetic polymer with unique crystalline structure when in condensed state or a highly reactive biopolymer such as amino-containing biopolymer such as chitosan and natural proteins such as albumin. No report of polymerized HA cryogel via crosslinking is disclosed therein. Also, there is no report on chemically derivatized biopolymer cryogels.

EP 1552839, EP 1005874, CN 1342722, JP 2000230002, JP 2003019194, JP 2000248002 and JP 2000230003 disclose methods of forming hyaluronic acid gels. None of these methods include the step of adding a cross-linking agent. The step of freezing a hyaluronic acid solution before less than 10% of the cross-linking bonds are formed is also not disclosed. This allows the formation of a relatively uniformly cross-linked hyaluronic acid product.

It is an object of the present invention to provide new HA based cryogels for biomedical and cosmetic applications, including regenerative medicine and tissue engineering.

It is another object of the present invention to provide cryogels prepared by chemical modification of HA in solution under freezing to a temperature below the solvent crystallization points. Such cryogels have improved properties relative to known macroporous polysaccharide hydrogels due to the mild reaction conditions.

It is still another object of the present invention to provide HA cryogels having properties particularly suited for their use in cosmetic surgery, wound dressing and post-surgical adhesion prevention.

It is another object of the present invention to provide HA cryogels which can be used as scaffolds for cell incorporation. The incorporated cells can be stem cells, fibroblasts cells, osteoblasts cells or chondrocytes for tissue repair.

It is also an object of the present invention to provide HA cryogels which can be produced in the form of an injectable gel, to pass through very fine needles, such as 18-30 G needles, as a tissue filler for cosmetic surgery.

It is still a further object of the present invention to provide HA cryogels in the form of rods, fibers, films, foams, cubes and spheres to be used in cosmetic surgery, for tissue augmentation, and tissue repair which is necessary due to injury caused by disease or external trauma.

It is still another object of the present invention to provide HA cryogels which can be produced in the forms above, to which pharmaceuticals or other bioactive substances can be incorporated for controlled drug delivery.

These and other objectives are attained by means of the present invention.

### DESCRIPTION OF THE INVENTION

According to a first aspect of the invention there is provided a process for preparing a HA cryogel, the process comprising the steps of:
a. combining a HA with defined molecule weight at suitable pH reaction condition, a cross-linking agent and a solvent to form a solution wherein if the HA has an average molecular weight of 500,000 or less the solvent is DMF, DMA or DMSO or the mixture of solvent with water. When water is present, the solvent includes alcohols;
b. cooling the solution to a temperature at least 5° C. below the solvent crystallisation point to form an at least partially-frozen solution; and
c. thawing the at least partially-frozen solution to provide a cross-linked polysaccharide cryogel,
   wherein: step b. is performed before the formation of less than 10% of the cross-linking bonds of the cross-linked HA cryogel formed.

According to the invention there is provided a process for preparing an HA cryogel, the process comprising the steps of:

a. combining an HA, a cross-linking agent and a solvent to form a solution;
b. cooling the solution to a temperature at least 5° C. below the solvent crystallisation point to form an at least partially-frozen solution; and
c. thawing the at least partially-frozen solution and purifying to provide a cross-linked HA cryogel,
wherein:
step b. is performed before the formation of less than 10% of the cross-linking bonds of the cross-linked HA cryogel formed.

It is known that HA and the like, degrade at low temperature. In particular, it is recommended that hyaluronic acid gels are not frozen in order to avoid structural degradation. As such, there is a prejudice in the art against subjecting HA to low temperatures.

Surprisingly, it has been found that using the process described above, hyaluronic acid (and its derivatives), can be cross-linked at or below the freezing point of several solvents or solvent systems to provide a cryogel. Degradation fragments are generally generated during known cross-linking methods, and this may be due to the temperatures employed. The presence of such degradation fragments adversely affects the yield and the biocompatibility of the cross-linked polysaccharide formed. The generation of such degradation products also means that well organised, uniformly cross-linked networks of polysaccharides are generally not formed through known cross-linking methods. In addition, the purification of the cross-linked polysaccharide to remove the degradation fragments is problematic. In addition the mechanical strength of known cross-linked polysaccharide hydrogels such as HA is generally limited; typically the strength (Young's module) is between 10-800 pa.

Whilst the inventors do not wish to be bound by any theory, it is believed that reducing the temperature of the solution reduces the rate of the cross-linking reaction and that this results in a uniformly cross-linked product comprising a network of cross-linked polysaccharide. In addition, the relatively low temperatures employed result in a reduction or prevention of the formation of degradation products. The strength of the HA cryogel formed according to the method of the present invention can match the highest Young's module of the current commercial products.

Surprisingly, contrary to the prejudice in the art, reducing the temperature of the HA during processing to form a cryogel does not result in structural degradation leading to high free HA (un-crosslinked HA).

The HA in this invention may have an average molecular weight of between 5,000 and 14,000,000 Daltons.

As noted above, where the HA has a low molecular weight, having an average molecular weight of 500,000 or less, a solvent of DMF, DMA, DMSO is used. Advantageously, the solvent is DMF. Typically the low molecular weight polysaccharide has an average molecular weight of between 5,000 and 500,000 Daltons, and more particularly between 10,000 and 100,000.

Alternatively HA may have a high molecular weight, having an average molecular weight of more than 500,000. Where a high molecular weight HA is used, any suitable solvent may be used including water, alcohols such as isopropanol, propylene glycol, ethanol or mixtures thereof. Typically the high molecular polysaccharide has an average molecular weight between 500,000 and 14,000,000 Daltons; particularly between 750,000 and 5,000,000 Daltons; more particularly between 750,000 and 2,000,000 Daltons.

Generally, the higher the molecular weight of HA, the greater the degree of cross-linking.

According to the method of the present invention, the temperature of the HA solution is cooled to at least 5° C. below the solvent crystallisation point before the formation of less than 10% of the cross-linking bonds of the cross-linked HA cryogel formed. Typically the HA solution is cooled before the formation of less than 5% of the cross-linking bonds of the cross-linked polysaccharide cryogel formed, suitably before the formation of less than 1% of the cross-linking bonds of the cross-linked HA cryogel formed.

As noted above, the temperature of the HA solution is cooled to at least 5° C. below the solvent crystallisation point, typically to at least 7° C. below the solvent crystallisation point, advantageously to at least 10° C. below the solvent crystallisation point.

The temperature to which freezing or chilling is carried out depends on the crystallization point of the solvent or solvent system used in each specific case. Said temperature should be at least 5° C. below the freezing point of the solvent or solvent system in order to keep the crystallization time down. For instance, in case of water as the solvent, freezing is generally carried out to a temperature within the range of from −5° C. to −40° C., preferably from −10° C. to −30° C.

Generally the temperature of the polysaccharide solution is cooled to −5° C. or less, typically to −20° C. or less, suitably to −50° C. or less, more suitably to about −65° C.

The solution is typically cooled from about 5° C. below the solvent crystallisation point to −196° C., being the boiling point of liquid nitrogen.

The solution may suitably be cooled to a temperature of between about 5° C. and about 40° C. below the solvent crystallisation point, and in particular the solution may be cooled to a temperature of between about 10° C. and about 30° C. below the solvent crystallisation point.

The process may comprise the step of mixing the solution to obtain a substantially homogenous mixture.

Preferably the process comprises the further step of adding a porogen to the solution. The porogen can be selected from the group consisting of: solvents such as alcohols, crystals such as sodium chloride, calcium carbonate.

Preferably the cross-linking agent is chosen from the group consisting of: polyepoxides; polyamines; dialdehydes; multifunctional amino acids; peptides in the presence of water-soluble carbodiimide; divinyl sulphone; and silicon-containing cross-linkers.

The polyepoxide can be chosen from the group consisting of: bisepoxybutane; ethyleneglycol diglycidyl ether; and bisepoxyoctane.

The polyamine can be chosen from the group consisting of: multi-arm PEG-amines; and polyethylene imines.

The dialdehyde can be chosen from the group consisting of: glyoxal; glutaraldehyde; and terephthalic aldehyde.

The silicon-containing cross-linker can be chosen from the group consisting of: tetraethoxylsilane; tetramethoxysilane; 3-aminopropyltriethoxysilane; 3-glycidoxypropyltrimethoxysilane; p-aminophenylsilane; n-(2-aminoethyl)-3-aminopropyltrimethoxysilane; 3-aminopropyltrimethoxysilane; 3-glycidoxypropyldiisonpropylethoxysilane; 3-glycidoxypropyltrimethoxysilane; 3-mercaptopropyltriethoxysilane; 3-mercaptopropyltrimethoxysilane; 3-methacryloxypropylmethyl diethoxysilane; 3-methacryloxypropyl trimethoxysilane; 3-isocyanatopropyltrimethoxysilane; 2-cyanoethyltriethoxysilane; tetraethyloxysilane; and tetramethyloxysilane. Optionally the cross-linking agent is chosen from the group consisting of: monoepoxides; monoamines; monoaldehydes; monovinyl-containing substances; and amino acids in the presence of carbodiimide.

The monoepoxide can be chosen from the group consisting of: epoxybutane; epoxyoctane; and epoxydecane.

The monoamine can be chosen from the group consisting of: mono PEG-amines; and aliphatic amines.

The monoaldehyde can be an aliphatic aldehyde.

The monovinyl-containing substance can be chosen from the group consisting of: vinyl-containing PEG; vinyl-containing acrylate; methyl methacrylate; and methacrylate.

Where the cross-linking agent comprises vinyl functional groups, the HA is suitably modified prior to formation of the solution. Typically the HA is modified to include vinyl functional groups prior to formation of the solution.

Typically the process of the present invention involves adding cross-linking agent in to HA solution with pH adjustment at low temperature. In contrast, prior art methods of cross-linking polysaccharides such as HA, generally at room temperature or above, comprise adding cross-linking agent into HA aqueous solution mainly.

In general, the greater the amount of cross-linking agent added, the greater the degree of cross-linking formed.

The HA cryogels of the present invention may be HA homopolymers or composites comprising more than one polymer.

The process may comprise the step of combining the HA, the cross-linking agent and the solvent with one or more monomers to form the solution. Typically the solution comprises one or more monomers such as vinyl monomers, for instance acryl amide, acrylic acid or acrylate.

Examples of monomers useful in the preparation of the HA hydrogels according to the invention are capable of forming crosslinked structure via polymerization. The monomers to be used in the preparation of the gels according to the present invention include but not limited to polyepoxides such as bisepoxybutane, ethyleneglycol diglycidyl ether, bisepoxyoctane etc, polyamines, typically as DNA carriers such as multiple arms PEG-Amines, polyethylene imines etc, dialdehydes such as glyoxal, glutaraldehyde, terephtalic aldehyde, etc, multifunctional amino acids and peptides in the presence of water-soluble carbodiimide, and divinyl sulphone. Silicon-containing crosslinkers such as tetraethoxylsilane, tetramethoxysilane, 3-aminopropyltriethoxysilane, 3-glycidoxypropyltrimethoxysilane, p-aminophenylsilane, n-(2-aminoethyl)-3-aminopropyltrimethoxysilane, 3-aminopropyltrimethoxysilane, 3-glycidoxypropyldiisonpropylethoxysilane, 3-glycidoxypropyltrimethoxysilane, 3-mercaptopropyltriethoxysilane, 3-mercaptopropyltrimethoxysilane, 3-methacryloxypropylmethyl diethoxysilane, 3-methacryloxypropyltrimethoxysilane, 3-isocyanatopropyltrimethoxysilane, 2-cyanoethyltriethoxysilane, TEOS, TMOS, etc.

Examples of monomers useful in the preparation of the hydrogels according to the invention are capable of forming derivatives via chemical derivatisation. The monomers to be used in the preparation of the gels according to the present invention include but not limited to mono-epoxides such as epoxybutane, epoxyoctane epoxydectane etc, amines, such as mono PEG-amine, aliphatic amine etc, aldehydes such as aliphatic aldehyde etc, mono-vinyl substance such as vinyl-containing PEG, vinyl containing acrylate such as methyl methacrylate, methacrylate etc, amino acid in the presence of carbodiimide etc.

Suitably the functional groups of the HA or HA derivatives polymerise with the monomers during the process of the present invention. The HA and the monomer(s) may polymerise together to form a cryogel comprising a uniform mixture of HA or HA derivative and monomer.

The HA derivatives may be containing additional functional group chosen from the group consisting of: amino, vinyl, aldehyde, thiol, silane, carboxyl, and hydroxyl.

Where the monomer comprises vinyl functional groups, the HA is suitably modified prior to formation of the solution. Typically the HA is modified to include vinyl functional groups prior to formation of the solution.

The further functionalisation of the HA prior to being cross-linked in accordance with the process of the present invention, promotes the polymerisation of the HA with the monomer. In particular, if the HA is functionalised with vinyl groups prior to the process of the present invention, polymerisation with the monomer is promoted.

Optionally the cryogel is further functionalised after thawing.

The cryogel may be further functionalised by the addition of a functional group chosen from the group consisting of: amino, vinyl, aldehyde, thiol, silane, carboxyl, and hydroxyl. Alternatively or in addition, the cryogel may be further functionalised by the addition of a further cross-linking agent.

Typically the further crosslinking agent is selected from the group consisting of polyepoxides such as bisepoxybutane, ethyleneglycol diglycidyl ether, bisepoxyoctane etc, polyamines, typically as DNA carriers such as multiple arms PEG-Amines, polyethylene imines etc, dialdehydes such as glyoxal, glutaraldehyde, terephtalic aldehyde, etc, multifunctional amino acids and peptides in the presence of water-soluble carbodiimide, and divinyl sulphone. silicon-containing crosslinkers such as tetraethoxylsilane, tetramethoxysilane, 3-aminopropyltriethoxysilane, 3-glycidoxypropyltrimethoxysilane, p-aminophenylsilane, n-(2-aminoethyl)-3-aminopropyltrimethoxysilane, 3-aminopropyltrimethoxysilane, 3-glycidoxypropyldiisonpropylethoxysilane, 3-glycidoxypropyltrimethoxysilane, 3-mercaptopropyltriethoxysilane, 3-mercaptopropyltrimethoxysilane, 3-methacryloxypropylmethyl diethoxysilane, 3-methacryloxypropyltrimethoxysilane, 3-isocyanatopropyltrimethoxysilane, 2-cyanoethyltriethoxysilane, TEOS and TMOS etc.

Optionally the process comprises the addition of two or more cross-linking agents to the solvent to form a multiple cross-linked network.

The solvent may be aqueous, and in particular may be acidic or alkaline.

The aqueous solvent may comprise a water-miscible solvent which can be chosen from the group consisting of: methanol, ethanol, dioxane, acetone, dimethyl sulfoxide, N,N-dimethylformamide, dimethylacetamide, and acetonitrile.

The solvent used in the process described is ordinarily water, but can also suitably be N,N-dimethylformamide (DMF) dimethylacetamide (DMA), or aqueous mixtures thereof. Also, the solvent used can be an alcohol such as isopropanol, propylene glycol, ethanol or aqueous mixtures thereof. The freezing points of these solvents, and their aqueous mixtures, can be found in the literature. However, for ease of reference, Table 1 below provides the freezing point of water when mixed with indicated quantities of isopropyl alcohol (IPA).

TABLE 1

| A Conc. Vol. % (Wt.) | Freezing Point, F. (° C.) |
|---|---|
| 0 (0) | 0 |
| 10 (8) | −4 |
| 20 (17) | −7 |
| 30 (26) | −15 |
| 40 (34) | −18 |
| 50 (44) | −21 |
| 60 (54) | −23 |
| 70 (65) | −29 |
| 80 (76) | −37 |
| 90 (88) | −57 |
| 100 (100) | <−73 |

Optionally the process comprises the further step of network densification of the cross-linked hydrogel during or after thawing. The network densification step may comprise the addition of a mixture of alcohol and acetone to the cross-linked hydrogel.

After the thawing step, the cryogel formed may be purified thoroughly with appropriate liquids (e.g. water, buffers, surfactant-containing solutions, organic solvents, mixtures of organic solvents with water or with each other) to remove impurities.

According to a preferred embodiment the cryogel may be further processed into an injectable gel, after thawing, by introducing further size reduction in order to pass the hydrogel through fine needles sized as 18-30 G.

In accordance with another embodiment of the hydrogel of the invention, the cryo-crosslinking and cryo-derivatisation can take place in a mould. After the thawing, the cryogels may be further purified without requiring de-mould.

The process may comprise the further step of casting the solution. Where the polysaccharide has a high molecular weight, the solution is typically cast into a mould having a desired shape, in particular the mould may be spherical, cubic or rod shaped.

Where the HA has a low molecular weight, the solution is typically cast or sprayed into a mould having a desired shape, in particular the mould may be spherical, cubic or rod shaped. Alternatively, the solution may be added to a solvent, such as alcohol, chloroform, acetone, said solvent having a temperature of −5 to −200° C., typically said solvent having a temperature of −5 to −196° C.

The pH conditions for the cryocrosslinking or cryo-derivatisation can be adjusted for preceding the reaction, which is well known to the persons skilled in the art. When epoxide agents are used, at pH<4, the reaction is mainly via carboxyl groups of the biopolymers, while at pH>8, the reaction is mainly via amino and hydroxyl groups.

When carbodiimide is used as crosslinking agent, pH needs to be controlled at neutral condition in the presence of amine or amino functional groups, or pH at 4 when ADH-modified HA is used or in the presence of other catalysts such as 1-hydroxybenzotriazole.

When aldehyde is used as crosslinking agent, pH needs to be controlled at acidic condition ranged from pH=2-6, preferably, at 2-4.

When vinyl sulphone is used as crosslinking agent, pH needs to be controlled at alkaline condition, pH>8, preferably, pH=9-11.

Optionally the process comprises the further step of adding a purification medium to the cross-linked hydrogel during or after thawing. The purification medium may comprise alcohol.

It has been found that low molecular weight hyaluronic acid can be cryo cross-linked more easily than bulky high molecular weight hyaluronic acid. To ensure a good cryogel formation with high molecular weight hyaluronic acid, it is beneficial to perform densification using a solvent during the thawing process.

The process of the present invention typically has a yield of more than 50%. In contrast, prior art methods of cross-linking polysaccharides such as HA, generally at room temperature or above, generally have a yield of less than 50%.

The process may comprise the further step of mixing the so-formed solution to form a substantially homogenous mixture.

In accordance with an example of embodiment of the method according to the present invention an aqueous solution of HA is crosslinked in the presence of polyepoxide crosslinking agent under freezing to the formation of a HA cryogel, thawing the HA cryogel, and then purified with phosphate saline to neutral and further processed into injectable macroporous hydrogel for tissue engineering.

The pH conditions for the cryocrosslinking or cryo-derivatisation can be adjusted for preceding the reaction, which is well known to the persons skilled in the art. When epoxide agents are used, at pH<4, the reaction is mainly via carboxyl groups of the biopolymers, while at pH>8, the reaction is mainly via amino and hydroxyl groups.

When carbodiimide is used as crosslinking agent, pH needs to be controlled at neutral condition in the presence of amine or amino functional groups, or pH at 4 when ADH-modified HA is used or in the presence of other catalysts such as 1-hydroxybenzotriazole.

When aldehyde is used as crosslinking agent, pH needs to be controlled at acidic condition ranged from pH=2-6, preferably, at 2-4.

When vinyl sulphone is used as crosslinking agent, pH needs to be controlled at alkaline condition, pH>8, preferably, pH=9-11.

It has surprisingly been found that using the process described above, hyaluronic acid (and its derivatives), can be cross-linked at or below the freezing point of several solvents or solvent systems to provide a cryogel. This is particularly surprising as HA contain relatively unreactive functional groups, such as carboxyl groups and hydroxyl groups, which would not be expected to cross-link at the reduced temperatures reported herein. The cryogels formed can be designed to have structural strength. Such materials can be used in cosmetic surgery, for tissue augmentation, and tissue repair. Alternatively, advantage can be taken of the porous nature of the materials in their use as carriers for pharmaceuticals or other bioactive substances for controlled drug delivery.

According to a second aspect of the invention there is provided a HA cryogel obtainable by the process as described herein.

Advantageously, the HA cryogel is formed from the method described above.

Typically the cryogel has a mechanical strength of between 10-800 pa in Young's Module; advantageously the cryogel has a mechanical strength of at least 800 pa while this is understood to be adjustable via the alteration of crosslinking conditions.

The formation of pores in the HA hydrogels described above is by virtue of the cryo-treatment during preparation. The pore size can range from nanopores to macropores (nanometre to micrometre), depending on the solvent used and the temperature applied, and typically the pore size is from 50 nm to 700 μm, and is advantageously from 500 nm to 700 μm. Alternatively the pore size is from 50 nm to 700 nm; typically 50 nm to 500 nm; suitably 100 nm to 500 nm. Generally at least 5% of the pores have the pore size specified above, typically at least 20%, suitably more than 50%, advantageously at least 70% of the pores have the pore size specified above. According to one embodiment the average pore size of the hydrogels of the present invention is as specified above.

The pore size can be controlled by the selection of the main solvent used, the incorporation and selection of a porogen (pore generating substance), the freezing temperature applied, the cross-linking conditions used, and also the molecule weight of HA.

The porogens can be particular solvents, crystalline particles or different sized salts such as sodium chloride. Alternatively, the pore size of the macroporous polysaccharide hydrogels can be controlled by incorporating additives of well-defined pore size. For example, solvent pre-filled silica can be incorporated into the cross-linking matrix after mixing and, and the mixture can be immediately exposed to cryo-conditions. After thawing, purification and drying, the pore size of the silica filler will be part of the porous structures for the macroporous polysaccharide hydrogels (or cryogel).

Generally at least 10% more of the volume of the pores of the cryogel of the present invention is contained within pores having a diameter of 100 nm to 700 nm compared to known hydrogels formed from the HA; more suitably at least 20% more of the volume of the pores of the cryogel of the present invention; advantageously at least 40% more of the volume of the pores of the cryogel of the present invention is contained within pores having a diameter of 100 nm to 700 nm compared to known hydrogels formed from HA.

Advantageously, the cryogel of the present invention comprises pores on its surface and this is in contrast to known hydrogels. Typically at least 10% of the surface of the cryogel of the present invention is contained within a pore.

It is widely acknowledged that tissue scaffolds comprising pores having a diameter of 100 to 500 nm promote and support the growth of tissue, such as bone, most effectively.

In contrast to known macroporous hydrogels formed from HA, the cryogel of the present invention typically comprises pores having a diameter of 100 to 500 nm, and thus can be used to effectively promote and support the growth of animal tissue. In addition the cryogel of the present invention has a higher mechanical strength than known macroporous hydrogels formed from the same polysaccharide. As such the cryogel of the present invention is useful in the promotion or augmentation of tissue growth. The cryogel of the present invention may be used to fill or bulk up tissue.

As noted above, known HA hydrogels generally comprise the degradation products of the polysaccharide and this may be due to the temperatures used to produce such hydrogel. Such degradation products include compounds such as water soluble HA fragments. In contrast, the cryogel of the present invention typically comprises less than 10% degradation products of the polysaccharide; advantageously less than 1%.

The cryogel of the present invention typically has a 90% of purity of crosslinked HA at least, and advantageously above 99% of purity of crosslinked HA.

The composition of the cryogel of the present invention can be predicted and determined with relative certainty as a relatively low percent of the cryogel is formed from such degradation products. As such, the properties of the cryogel of the present invention can be predicted with relative certainty. As the make-up of the cryogel of the present invention is largely predictable, and the purity of the cryogel of the present invention is relatively high, the cryogel is biocompatible.

The cryogel of the present invention is generally more stable. This may be due in part to the relatively low amounts of degradation product contained within the cryogel. This stability of the cryogel of the present invention means that it is particularly suitable for applications such as promoting and supporting animal tissue growth and that the cryogel of the present invention is suitable for implantation in a human or animal body.

The pH of the HA hydrogel is typically 6 to 8, suitably 6.5 to 7.5, more suitably 6.5 to 7.4

The viscosity of the HA hydrogel depends on its intended use. However, the complex viscosity is generally at least 50 pa·s at 0.01 Hz (frequency), typically at least 100 pa·s at 0.01 Hz, suitably at least 150 pa·s at 0.01 Hz.

Typically the HA cryogel of the present invention is non-cytotoxic.

The HA hydrogel according to the present invention may be in the shape of a rod, cube and sphere. It also can be in the form of a film or a foam. Alternatively, the HA hydrogel according to the invention is prepared in the form of particles.

The particles can be formed by spraying or dropping into freezing medium such as alcohol medium to precede the cryocrosslinking process.

Alternatively, the particles can be formed by further down-stream size reduction process after the thawing.

The HA cryogel described herein can be further chemically treated or derivatised to enhance their mechanical properties or to produce tissue functionality. For example, they can be modified to immobilize growth factors, DNA, enzymes, or peptides which can enhance tissue adhesion or promote cell attachment and proliferation. In addition, the formed hydrogel can be mixed with cells to provide tissue engineered products, or can be used as a bio-matrix to aid tissue repair or tissue augmentation (for example, breast implants or tissue fillers). According to one aspect of the present invention the HA cryogel is in the form of a drug delivery matrix.

The HA cryogels described herein can also be mixed with pharmaceuticals for drug delivery for clinical applications such as pain control, cancer treatment, wound care treatment or anti-infection.

According to a further aspect of the present invention there is provided the HA cryogel for use as a medicament.

According to a further aspect of the present invention there is provided the use of the HA cryogel for use in tissue augmentation, surgical applications (including cosmetic surgery and vascular surgery), cardiology, ophthalmology, orthopaedics, wound dressing, post surgical adhesion prevention, regenerative medicine applications, transcatheter, ENT applications, tissue engineering applications or for use as a scaffold for the incorporation of cells such as a scaffold for the incorporation of cells such as stem cells, fibroblast cells, osteoblast cells or chondrocytes for tissue repair.

According to a further aspect of the present invention there is provided a method of medical treatment comprising the steps of administering the cryogel of the present invention for tissue augmentation, cosmetic surgery, wound dressing, post surgical adhesion prevention, regenerative medicine applications, tissue engineering applications or for use as a scaffold for the incorporation of cells such as stem cells, fibroblast cells, osteoblast cells or chondrocytes for tissue repair.

According to a further aspect of the invention there is provided a material for use in tissue augmentation, the material comprising a polysaccharide cryogel obtainable by the process described herein.

When used in the field of tissue augmentation, the hydrogel of the present invention generally has a complex viscosity of 100 pa·s at 0.01 Hz (frequency) or more, a pH of 6.5 to 7.4. Generally at least 5% of the pores of such a hydrogel have a pore size of 50 nm to 500 nm; typically at least 20% of the pores, suitably at least 50% of the pores, advantageously at least 80% of the pores have a pore size of 50 nm to 500 nm. The average pore size of the hydrogel of the present invention is preferably 50 nm to 500 nm when used in the field of tissue augmentation.

The material may be a dermal filler.

According to a further aspect of the invention there is provided an injectable particulate composition comprising a HA cryogel obtainable by the process described herein.

According to a still further aspect of the invention there is provided a wound dressing comprising a HA cryogel obtainable by the process described herein.

According to a further aspect of the invention there is provided a drug delivery composition comprising a HA cryogel obtainable by the process described herein.

The drug delivery composition typically comprises pharmaceutical or bioactive substances.

According to a still further aspect of the invention there is provided an anti-aging composition comprising a HA cryogel obtainable by the process described herein.

In accordance with a further aspect of the present invention there is provided the use of the cryogel according to the present invention in the field of tissue engineering and regenerative medicine, particularly, in the field of cosmetic surgery for tissue augmentation in the form of particles, rods, spheres and cubes.

According to this aspect of the present invention there is also provided the use of the cryogel according to the present invention in the field of wound dressing for promote wound healing in the forms of gels, film, fibres and foams.

According to this aspect of the present invention there is also provided the use of the cryogel according to the invention for incorporation cells for tissue repair and regeneration.

According to this aspect of the present invention there is also provided the use of the cryogel according to the invention for incorporation of pharmaceutical substances to achieve controlled delivery. The pharmaceutical substances include but not limited to antibiotics, antiseptics, anesthetics, anticancer drugs, proteins, growth factors etc.

Further according to this aspect of the present invention there is also provided the use of the cryogel according to the invention for the application in post-surgical adhesion prevention when gels and films are applied.

According to one aspect of the present invention there is provided a HA cryogel obtainable by mixing an aqueous solution of HA, chemically containing functional groups such as carboxyl, hydroxyl, amino or further functionalized to contain vinyl, aldehyde, thiol, silane; and monomers capable of reacting with above groups via chemical derivatisation or chemical crosslinking; and other components such as nonaqueous solvent or water miscible solvent and fillers to manipulate the pore structures; under freezing at a temperature below the solvent crystallization point, at which solvent in the system is partially frozen with the dissolved substances concentrated in the nonfrozen fraction of solvent to the formation of a cryogel.

In one or more steps by methods known per se subsequent to thawing of the cryogel, purify and densify the gel or carrying out the process in a mould with defined three-dimension to achieve different shape, after thawing of the cryogel, modifying the cryogel as set forth under above.

The invention will now be further exemplified with reference to the accompanying Figures in which:

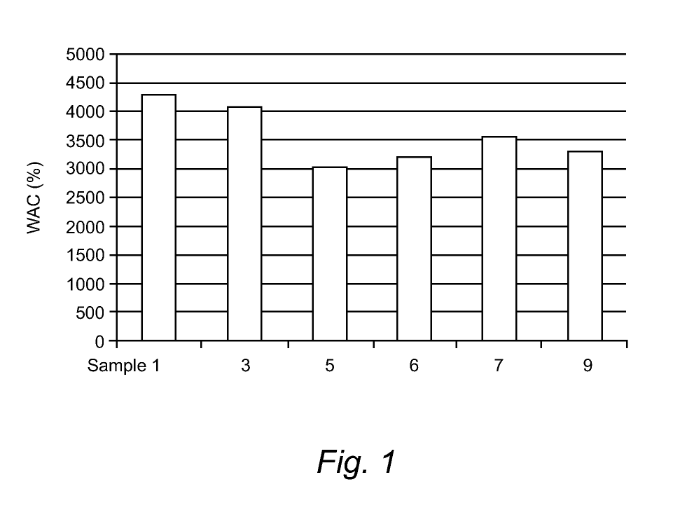

FIG. 1 illustrates the effect of molecular weight on the degree of cross-linking of the HA cryogel;

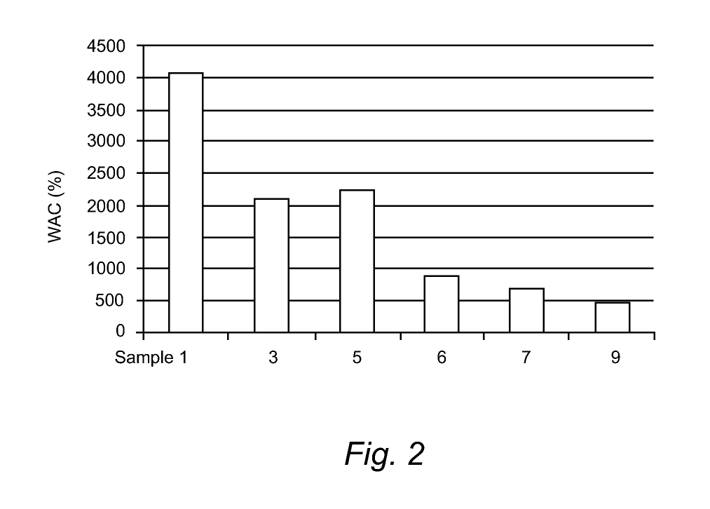

FIG. 2 illustrates the effect of molar ratio of HA/cross-linking agent on the degree of cross-linking of the HA cryogel;

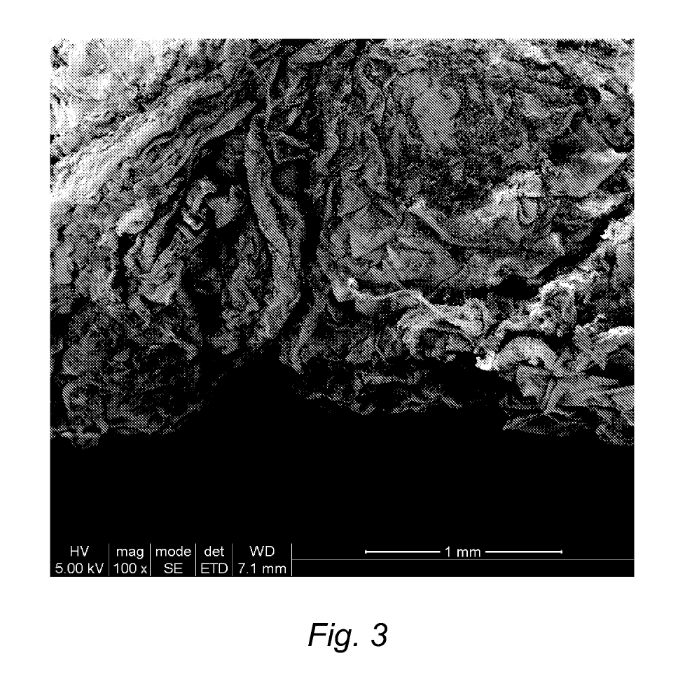

FIG. 3 shows a SEM image of cross-linked HA at 1 mm scale;

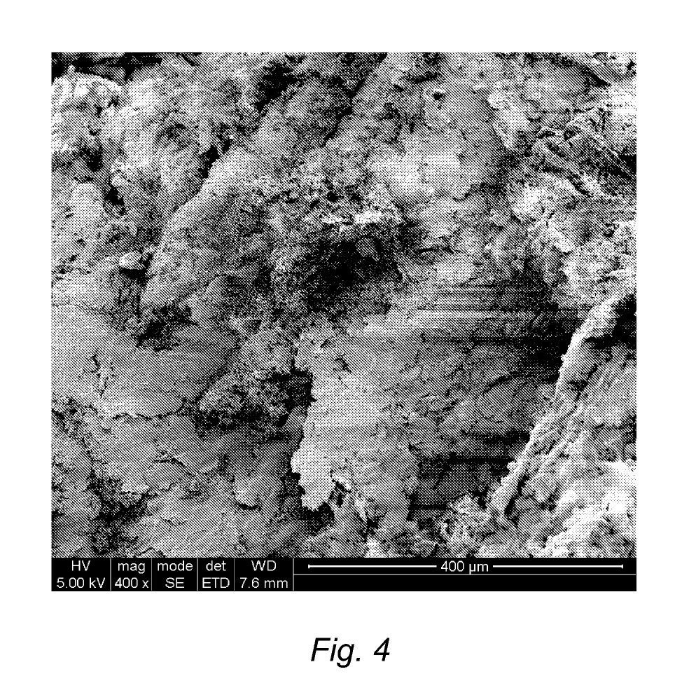

FIG. 4 shows a SEM image of cross-linked HA at 400 micron scale;

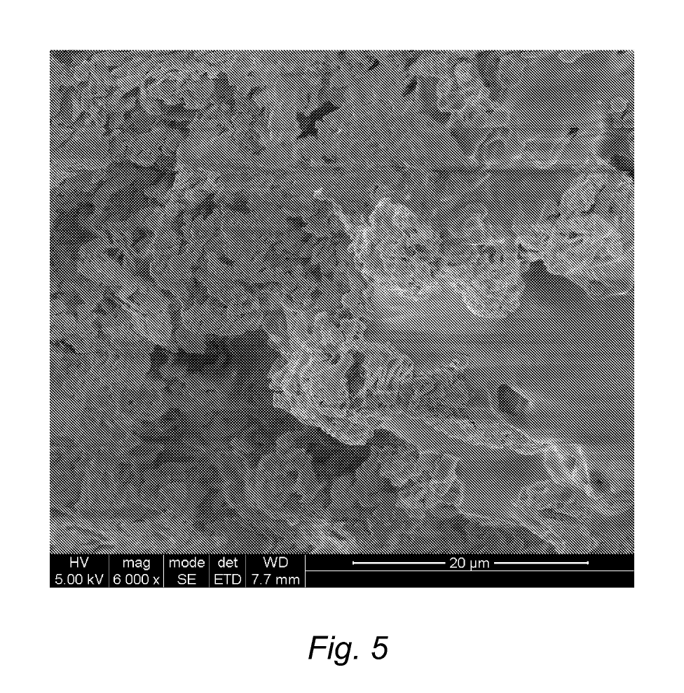

FIG. 5 shows a SEM image of cross-linked HA at 20 micron scale showing micropores and macropores;

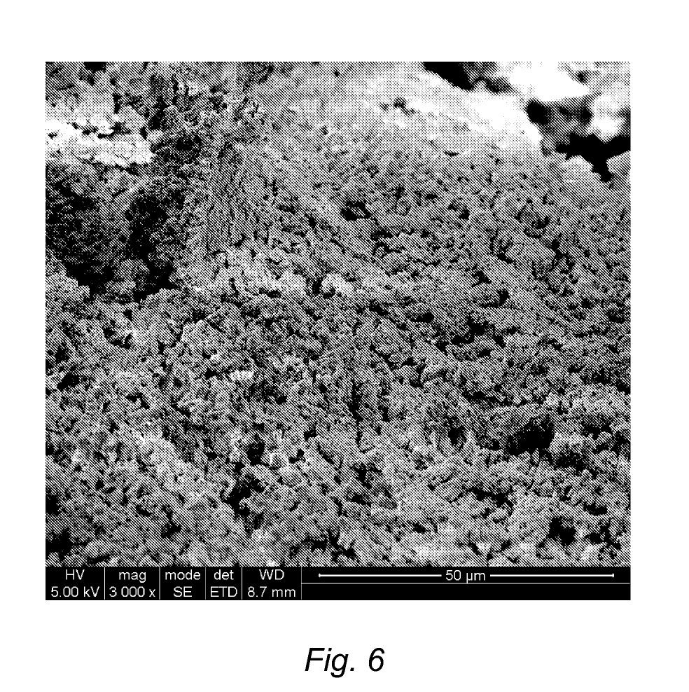

FIG. 6 shows a SEM image of cross-linked HA at 50 micron scale showing micropores and macropores;

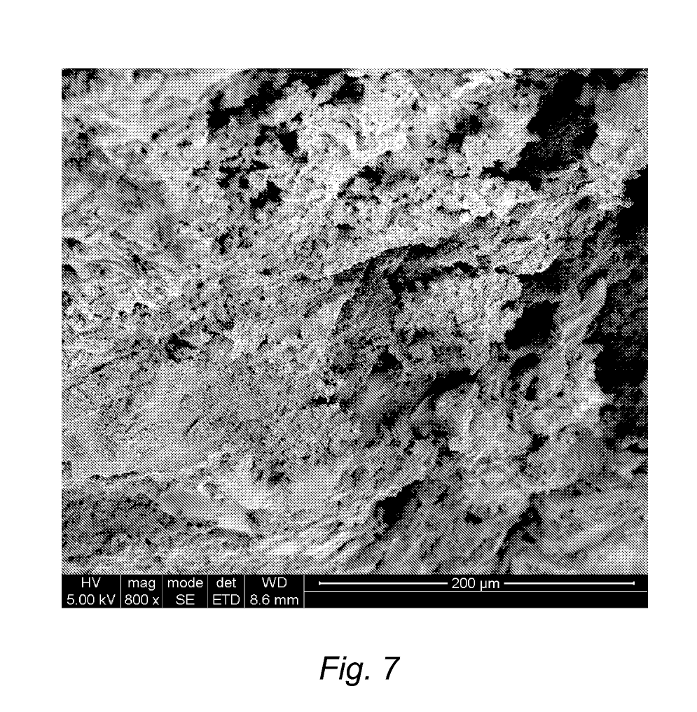

FIG. 7 shows a SEM image of cross-linked HA at 200 micron scale showing macropores;

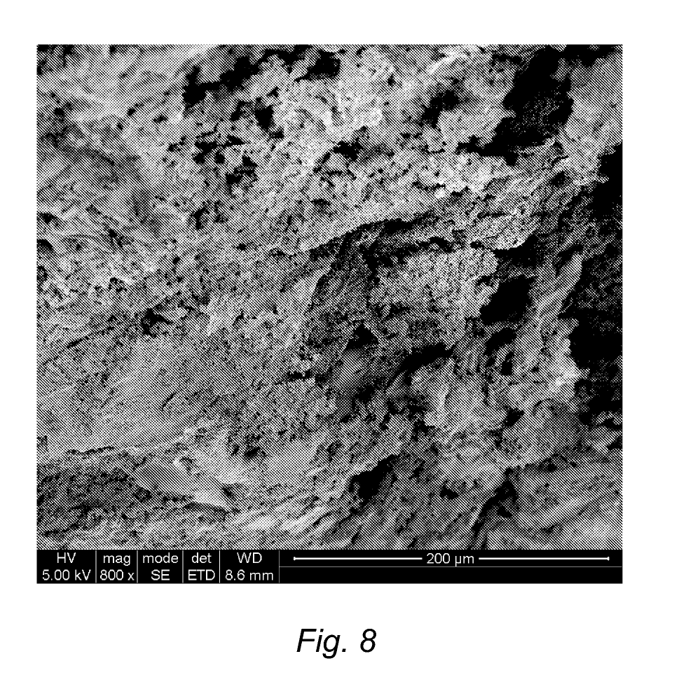

FIG. 8 shows a SEM image of cross-linked HA at 200 micron scale showing macropores

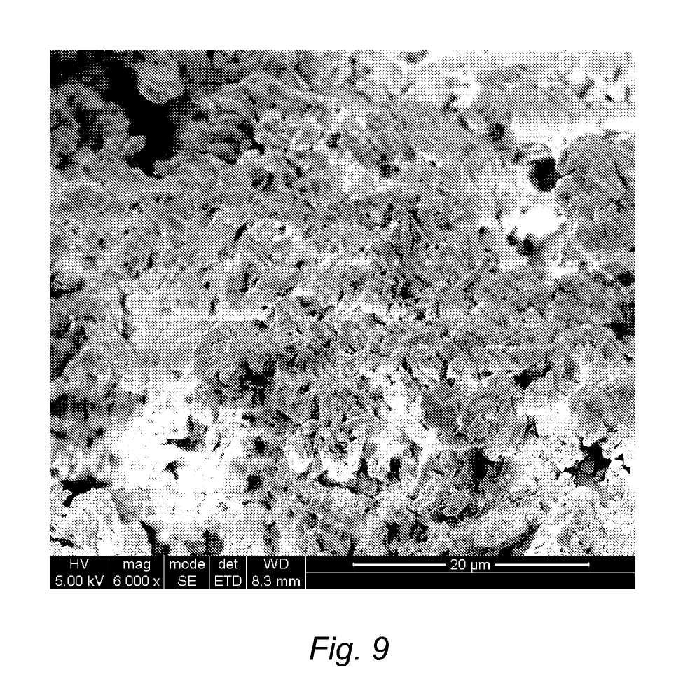

FIG. 9 shows a SEM image of cross-linked HA at 20 micron scale showing micropores and macropores;

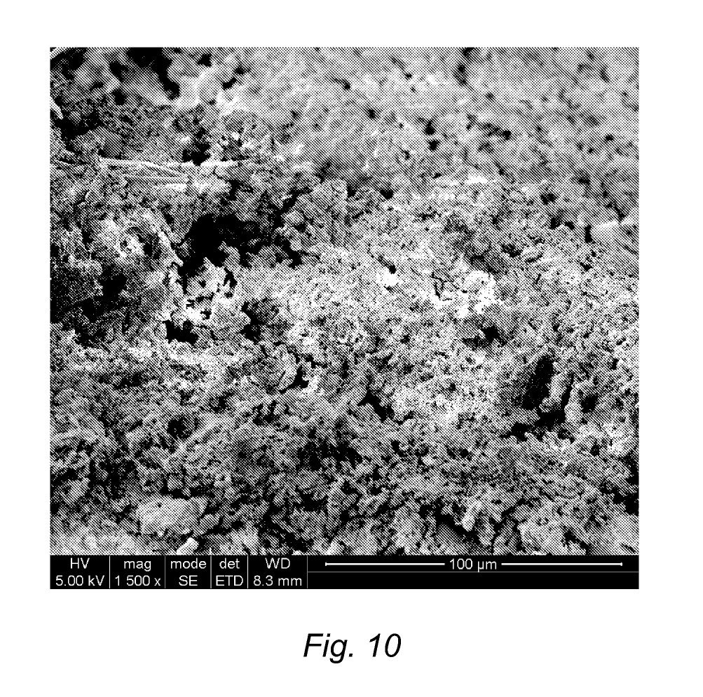

FIG. 10 shows a SEM image of cross-linked HA at 100 micron scale showing micropores and macropores;

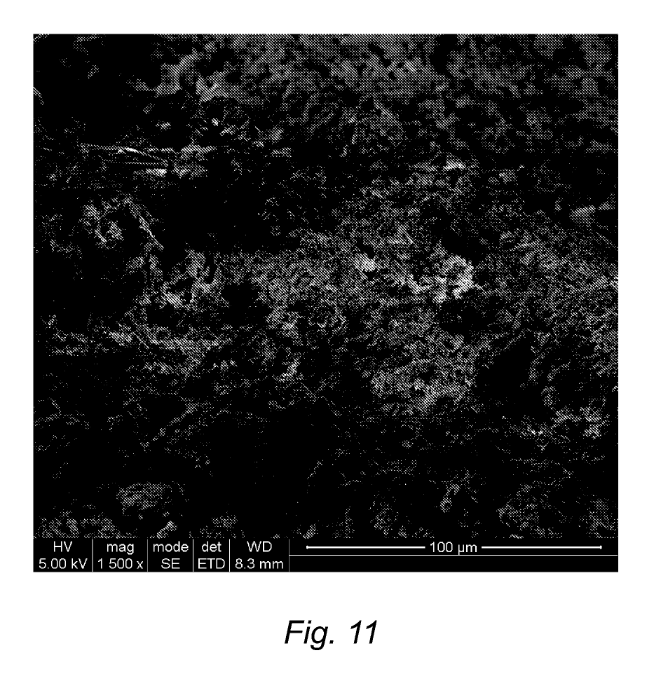

FIG. 11 shows a SEM image of cross-linked HA at 100 micron scale showing micropores and macropores;

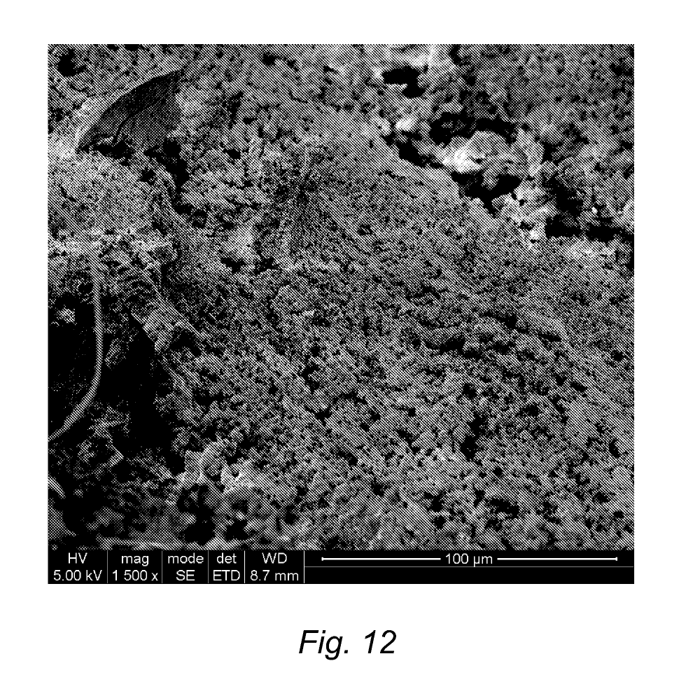

FIG. 12 shows a SEM image of cross-linked HA at 100 micron scale showing micropores and macropores;

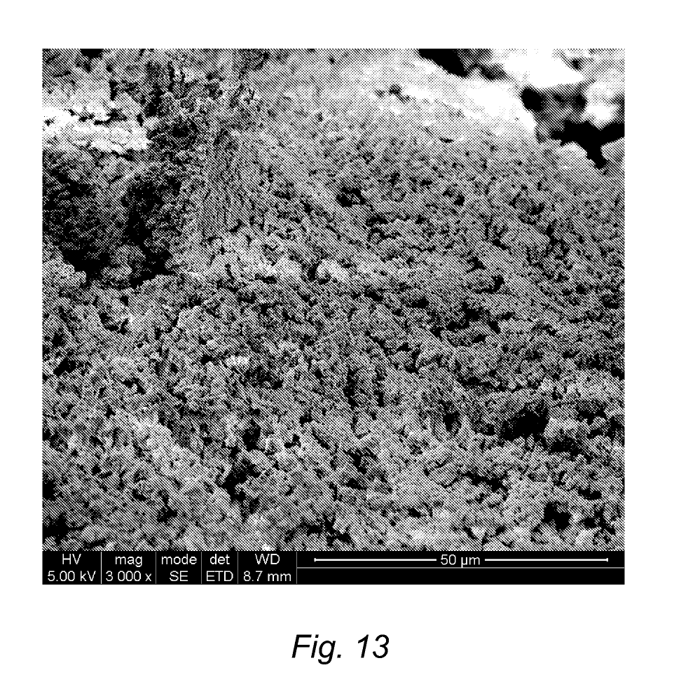

FIG. 13 shows a SEM image of cross-linked HA at 50 micron scale showing micropores and macropores;

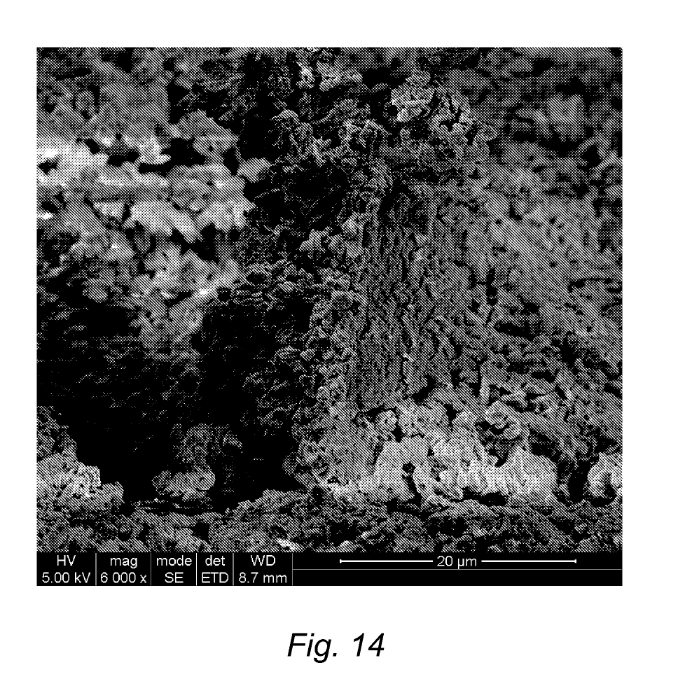

FIG. 14 shows a SEM image of cross-linked HA at 20 micron scale showing micropores and macropores.

### EXAMPLE 1

### Preparation of the HA Cryogel

The solution of HA (MW 6000) (10 g), sodium hydroxide (2 g) in 60 ml of DMF/H2O (40/20) was degassed and after addition of 1 ml of BDDE and have a good mixing was slowly poured into a glass tube. The polymer solution in the tube was frozen and kept at −18° C. overnight. Then the tube was thawed at IPA and a spongy cryogel thus formed was washed thoroughly with IPA to remove the impurities and vacuum dried to obtain HA cryogel.

### EXAMPLE 2

### Preparation of the HA Cryogel

The solution of HA (MW 20,000) (10 g)), sodium hydroxide (2 g) in 60 ml water was degassed and after addition of 1 ml of BDDE and have a good mixing was slowly poured into a glass tube. The polymer solution in the tube was frozen and kept at −18° C. overnight. Then the tube was thawed at IPA and a spongy cryogel thus formed was washed thoroughly with IPA to remove the impurities and vacuum dried to obtain HA cryogel.

### EXAMPLE 3

### Preparation of the HA Cryogel

The solution of HA (MW 1,000,000) (10 g)), sodium hydroxide (2 g) in 60 ml water was degassed and after addition of 1 ml of BDDE and have a good mixing was slowly poured into a glass tube. The polymer solution in the tube was frozen and kept at −18° C. overnight. Then the tube was thawed and a spongy cryogel thus formed was washed thoroughly with IPA to remove the impurities and vacuum dried to obtain HA cryogel.

### EXAMPLE 4

### Preparation of HA Cryogel

The solution of HA (MW 1,000,000) (10 g), sodium hydroxide (2 g), 5 g of sodium chloride in 60 ml water was degassed and after addition of 1 ml of DVS and have a good mixing was slowly poured into a glass tube. The polymer solution in the tube was frozen and kept at −18° C. overnight. Then the tube was thawed and a spongy cryogel thus formed was washed thoroughly with IPA to remove the slats and impurities and vacuum dried to obtain HA cryogel.

### EXAMPLE 5

### Preparation of HA Cryogel

The solution of HA (MW 100,000) (10 g), sodium hydroxide (2 g) in 60 ml water was degassed and after addition of 5 ml of glycidyl methacrylate and mixed to react for 8 hours. Neutralize the solution with acid to stop the reaction and to the mixture, N,N-methylene-bis-acrylamide (1 g) and N,N, N′,N′-tetramethylenediamine (TEMED 1 g) and 50 mg of solid ammonium persulfate were added and have a good mixing. The mixture was slowly poured into a glass tube. The polymer solution in the tube was frozen and kept at −18° C. overnight. Then the tube was thawed and a spongy cryogel thus formed was washed thoroughly with IPA to remove the impurities and vacuum dried to obtain HA cryogel.

### EXAMPLE 6

### Preparation of HA Cryogel

The solution of HA (MW 50,000) (10 g), 4 g sodium hydroxide in 60 ml water was degassed and after addition of 3-glycidoxypropyltrimethoxysilane and had a good mix and slowly poured into a glass tube. The polymer solution in the tube was frozen and kept at −18° C. overnight. Then the tube was thawed and a spongy cryogel thus formed was washed thoroughly to remove the impurities. A silicon-containing crosslinked HA Hydrogel is formed.

### EXAMPLE 7

### Preparation of HA Cryogel

The solution of HA (MW 10,000) (10 G), dissolved in 20 g DMF, transfer the solution into a 20 g of purified water, to it 2 g of sodium hydroxide was added, and after addition of epoxyoctane (sigma) (2 G) and had a good mix and slowly poured into a glass tube. The polymer solution in the tube was frozen and kept at −18° C. overnight. Then the tube was thawed and a spongy cryogel thus formed was washed thoroughly to remove the impurities. A hydrophobe derivatised HA Hydrogel is formed. The material can be further crosslinked using BDDE in the suspension of solvent/water.

### EXAMPLE 8

### Preparation of HA/DNA Hydrogel

The solution of HA (MW 50,000) (10 g) and 1.0 g DNA (sigma), in 60 ml water was degassed and after addition of 0.1 g glutaraldehyde and had a good mix and slowly poured into a glass tube. The polymer solution in the tube was frozen and kept at −18° C. overnight. Then the tube was thawed and a spongy cryogel thus formed was washed thoroughly to remove the impurities. A crosslinked HA/DNA Hydrogel is formed.

### EXAMPLE 9

### Applications of Crosslinked HA/DNA Cryogel for Topical Application

Mix 1.0 g of material obtained from example 8 with PVA cosmetic formula to form a film-forming material for skin-peel as anti-wrinkle cosmetic.

### EXAMPLE 10

### Formulation of Crosslinked HA Gel for Tissue Augmentation

10 g of fully swollen cross-linked HA gel was suspended in 1 L phosphate buffered solution and homogenised with a homogeniser. After filtration with sieves, gel comprising particles sized between 200 and 300 micron able to pass through a 30 G needle was obtained. The gel particles were filled into syringes and steam sterilized through to obtain sterile gel. The sterilized gel was suitable for use in tissue augmentation applications.

### EXAMPLE 11

### Formulation of Crosslinked HA Gel Containing Lidocaine for Tissue Augmentation

10 g of fully swollen cross-linked HA gel was suspended in 1 L phosphate buffered solution and homogenised with a homogeniser. After filtration with sieves, gel comprising particles sized between 200 and 300 micron to pass through a 30 G needle was obtained. Lidocaine was added to the gel to obtain a gel containing 0.3% lidocaine. The gel was filled into syringes and steam sterilized to obtain sterile gel. The sterilized gel was suitable for use in tissue augmentation applications.

### EXAMPLE 12

### Formulation of Crosslinked HA Gel for Wound Dressing

Cross-linked HA gel with a different degree of cross-linking (with variable water absorbance capacity) was milled into fine gels. The materials were autoclaved and filled into tubes for wound dressing gels to promote the wound healing process.

### EXAMPLE 13

Crosslinked HA gel was mixed with fibroblasts cells or Chondyocytes cells to form a gel medium. The gel can be injected into dermis for tissue augmentation or cartilage repair in regenerative medicine.

### EXAMPLE 14

### Application of Crosslinked HA Cryogel for Tissue Augmentation

Homogenise samples obtained in Example 4/5 to obtain particle size around 100 micron and suspend in phosphate buffered saline and sterilized terminally to be able to pass through 30 G needle. Inject 0.5 ml material subcutaneously into a rabbit animal model and the material can persist in-situ for 9-12 months.

### EXAMPLE 15

### Effect of Molecular Weight of HA on Water Absorption Capacity

HA cryogel of the present invention was prepared using HA of varying molecular weight. The molecular weight of the HA varied from less than 200,000 to 2 million as detailed in Table 2 below. The degree of cross-linking of the HA cryogel was assessed by assessing the water absorption capacity (WAC) of the HA cryogel. The WAC of the cryogels was calculated according to Formula A below:

WAC(%)=(Wgel−W0)/W0×100

Where W0=initial weight
Wgel=the weight of the gel after immersion in purified water for 24 hours

TABLE 2

| Sample No | MW of HA | Wo | Wgel | WAC (%) |
|---|---|---|---|---|
| 1 | W-200,000 | 0.24 | 10.57 | 4304 |
| 3 | W-500,000 | 0.27 | 11.35 | 4104 |
| 5 | W-1 million- | 0.2 | 6.3 | 3050 |
| 6 | W-1 million | 0.27 | 8.94 | 3211 |
| 7 | W-1 million | 0.28 | 10.26 | 3564 |
| 9 | MW-2 million | 0.28 | 9.55 | 3311 |

From the results, it can be seen that LMW HA will result in less degree of crosslinking at the same crosslinking ratio as those HMW HA while there is no difference between 1 million and 2 million MW HA.

It was found that use of low molecular weight HA resulted in less cross-linking than the use of high molecular weight HA. However, there was little change in the degree of cross-linking between HA having a molecular weight of 1000000 and 2000000. This is illustrated in FIG. 1.

### EXAMPLE 16

### Effect of Molar Ratio of HA/Cross-Linking Agent on Water Absorption Capacity (WAC)

HA cryogel of the present invention was prepared using HA having a molecular weight of 1,000,000. A BDDE cross-linking agent was used in the preparation of the HA cryogel. The molar ratio of HA/cross-linking agent was varied as shown in Table 3 below. The degree of cross-linking of the HA cryogel was assessed by assessing the water absorption capacity (WAC) of the HA cryogel.

TABLE 3
Effect of HA/BDDE molar ratio on the WAC (%) of crosslinked HA

| | HA/BDDE | | | |
|---|---|---|---|---|
| Samples | molar ratio | Wo | Wgel | WAC (%) |
| 1 | 10/1 | 0.5 | 20.82 | 4064 |
| 3 | 5/1 | 0.41 | 9.02 | 2100 |
| 5 | 4/1 | 0.33 | 7.69 | 2230 |
| 6 | 2/1 | 0.31 | 3.08 | 894 |
| 7 | 1/1 | 0.38 | 2.94 | 674 |
| 9 | 1/2 | 0.4 | 2.26 | 465 |

As a hydrogel, the WAC gives an indication of the degree of crosslinking, with a high degree of crosslinking normally leading to a low WAC. This clearly indicates the more BDDE added, the higher the degree of crosslinking resulting in less water absorption capacity. This also means the crosslinked HA can be adjusted to have different degree of crosslinking to suit different needs in terms of biodegradation rate in the body for tissue engineering.

It was found that use of a low HA/cross-linking agent molar ratio resulted in a greater degree of cross-linking than a high HA HA/cross-linking agent molar ratio. This is illustrated in FIG. 2.

### EXAMPLE 17

### Cytotoxicity Assessment to Verify Biocompatibility of HA Cryogel

HA by nature is of excellent biocompatibility. However, by modification, the biocompatibility can be altered due to the change of the chemical structures. The extreme modification can change the conformation of HA which leads to some cyto-incompatibility.

In order to demonstrate the crosslinked HA under above conditions have no effect on the cell lysis or any toxic effects at cells level, the standard cytotoxicity assessment was carried out on one of the crosslinked HA materials by a sub-contractor testing house.

### Methods

### Test System Management and Justification

Mammalian cell culture monolayers, L929, mouse fibroblast (ATCC CCL1, NCTC clone 929, of strain L, or equivalent source) were used. In vitro mammalian cell culture studies have been used historically to evaluate cytotoxicity of biomaterials and medical devices, and L929 cells are recommended by the ISO 10993-5 standard.

These cells were propagated at 37° C. in a gaseous environment of 5% carbon dioxide (CO₂) in an open flask containing Minimum Essential Medium Eagle 1X (EMEM1X) supplemented with 10% foetal bovin serum (v/v), 1% L-Glutamine (v/v) and an appropriate concentration of antibiotics (penicillin-streptomycin 2% (v/v)). For this study, 35 mm wells were seeded, labelled with passage number and date, and incubated at 37° C. in 5% CO₂ in order to obtain confluent monolayers of cells prior to use. Aseptic procedures were used in the handling of the cell cultures following approved BIOMATECH Procedures (I-BIO 080).

### Preparation of Agar Overlay

Equal amounts of double strength Minimum Essential Eagle 2X (EMEM2X) and 3% agarose (v/v) were combined to form an EMEM2X-agarose mixture (final dilution 1.5% agarose, 1×EMEM (v/v)).

Monolayers of L-929 mouse fibroblast cell cultures were grown to confluency in culture wells. The EMEM-agarose mixture (x mL) was then placed in the culture wells and allowed to solidify over the cells to form the agarose overlay.

### Experimental Procedures

Triplicate culture wells, which contained a confluent cell monolayer were selected. About 1 cm² pieces of the test article, negative and positive controls were each placed directly onto triplicate solidified overlay surfaces. Each well was labelled indicating its contents and incubated at 37° C. in 5% CO₂ for 24-26 hours.

Following incubation, the cell cultures were stained by a neutral red solution and macroscopically examined for cell decolorization around the pieces of the test article, negative and positive controls to determine the zone of the cell lysis (if any). After macroscopic examination, the cell monolayer were microscopically examined (at least 100×) to verify any decolorized zones and to determine cell morphology in proximity and beneath the test and control articles.

Scoring for cytotoxicity was based on the following criteria:

ASTM F 895-84 (reapproved 2006)

### Standard Test Method for Agar Diffusion Cell Culture Screening for Cytotoxicity

### Annual Bood of ASTM Standard

$$Response index = \frac{Zone index ( 0 \text{-} 5 )}{Lysis index ( 0 \text{-} 5 )}$$

TABLE 1
determination of the zone index

| ZONE INDEX | ZONE DESCRIPTION |
|---|---|
| 0 | no detectable zone around and under |
| | specimen |
| 1 | zone limited to area under specimen |
| 2 | zone extending less than 0.5 cm beyond |
| | specimen |
| 3 | zone extending from 0.5-1 cm beyond |
| | specimen |
| 4 | zone extending more than 1 cm beyond |
| | specimen but does not involve entire dish |
| 5 | zone involves the entire dish |

TABLE 2
determination of the lysis index

| LYSIS INDEX | LYSIS DESCRIPTION |
|---|---|
| 0 | no observable cytotoxicity |
| 1 | less than 20% of zone affected |
| 2 | 20 to 39% of zone affected |
| 3 | 40 to 59% of zone affected |
| 4 | 60 to 80% of zone affected |
| 5 | greater than 80% of zone affected |

For the suitability of the system to be confirmed, the negative controls must have been a zone index of 0 and a lysis index of 0, i.e. a response index of 0/0. The positive control must have produced a zone index of a least 1 and a lysis index of 5 i.e. a response index of at least 1/5.

The test article met the requirements of the test if neither of cell cultures exposed to the test article showed greater a response index of 0/0. The test would have been repeated if the controls did not perform as anticipated and/or one of the three test wells did not yield the same conclusion.

### Results

| | ZONE | LYSIS | RESPONSE |
|---|---|---|---|
| | INDEX | INDEX | INDEX |
| Test article | 0 | 0 | 0/0 |
| | 0 | 0 | 0/0 |
| | 0 | 0 | 0/0 |
| Positive control | 3 | 5 | 3/5 |
| | 3 | 5 | 3/5 |
| | 3 | 5 | 3/5 |
| Negative control | 0 | 0 | 0/0 |
| | 0 | 0 | 0/0 |
| | 0 | 0 | 0/0 |

Results and conclusions apply only to the test article tested. No further evaluation of these results is made by BIOMATECH. Any extrapolation of these data to other samples is the responsibility of the Sponsor.

### CONCLUSION

Under the conditions of this study, the test article showed no evidence of cell lysis or toxicity greater than a response index of 0/0, test article response index of 0/0. The test article met the requirements of the test. The negative and positive controls performed as anticipated.

This preliminary result demonstrates the crosslinked HA under cryo-condition won't have adverse effects on cell growth to show the cell biocompatibility.

Various modifications and variations to the described embodiments of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A process for preparing a HA cryogel, the process comprising the steps of:
a. combining HA, a cross-linking agent and a solvent to form a solution wherein if the HA has an average molecular weight of 500,000 or less the solvent is DMF, DMA, DMSO, water, water/solvent mixtures;
b. cooling the solution to a temperature at least 5° C. below the solvent crystallisation point to form an at least partially-frozen solution; and
c. thawing and purification of the at least partially-frozen solution to provide a cross-linked polysaccharide cryogel,
wherein:
step b. is performed before the formation of less than 10% of the cross-linking bonds of the cross-linked HA cryogel formed.

2. The process of claim 1, wherein the HA has an average molecular weight of 10,000 and 100,000 Daltons.

3. The process of claim 1, wherein the HA has an average molecular weight of 750,000 and 2,000,000 Daltons, and the solvent is aqueous or alcoholic.

4. The process of claim 1, wherein the temperature of the HA solution is cooled to at least 5° C. below the solvent crystallisation point before the formation of less than 1% of the cross-linking bonds of the cross-linked HA cryogel formed.

5. The process of claim 1, wherein the temperature of the HA solution is cooled to between 10 to 30° C. below the solvent crystallisation point.

6. The process of claim 1, wherein the temperature of the HA solution is cooled to between −65 to −196° C.

7. The process of claim 1, comprising the further step of adding a porogen to the solution.

8. The process of claim 7, wherein the porogen is selected from the group consisting of: solvents such as alcohols, acetone, crystals such as sodium chloride, calcium carbonate.

9. The process of claim 1, wherein the cross-linking agent is selected from the group consisting of: polyepoxides; polyamines; dialdehydes; multifunctional amino acids; peptides in the presence of water-soluble carbodiimide; divinyl sulphone; and silicon-containing cross-linkers.

10. The process of claim 1, wherein the cross-linking agent is selected from the group consisting of: monoepoxides; monoamines; monoaldehydes; monovinyl-containing substances; and amino acids in the presence of carbodiimide.

11. The process of claim 1, wherein the solution comprises one or more monomers.

12. The process of claim 11, wherein the monomer(s) is/are selected from the group consisting of acryl amide, acrylic acid or acrylate.

13. The process as claimed in claim 1, wherein the HA is further functionalised by the addition of a functional group chosen from the group consisting of: amino, vinyl, aldehyde, thiol, silane, carboxyl, and hydroxyl functional groups.

14. The process as claimed in claim 11, wherein the monomer comprises vinyl functional groups, and the HA is modified to include vinyl functional groups prior to formation of the solution.

15. The process as claimed in claim 1, comprising the step of network densification of the cross-linked hydrogel during or after thawing.

16. The process as claimed in claim 15, wherein the cross-linked HA cryogel formed is further functionalised by the addition of a further cross-linking agent.

17. The process as claimed in claim 1, wherein two or more cross-linking agents are added to the solution to form a multiple cross-linked network.

18. The process as claimed in claim 1, comprising the step of casting the solution.

19. The process as claimed in claim 1, comprising the step of adding a purification medium to the cross-linked polysaccharide cryogel during or after thawing.

20. The process as claimed in claim 1 having a yield of at least 50%.

21. A HA cryogel obtainable by the process as claimed in claim 1.

22. A HA cryogel comprising a porous cross-linked network, wherein the HA cryogel is at least 10% porous and at least 10% of the volume of the pores of the polysaccharide cryogel are contained within pores having a diameter of from about 50 nm to about 700 nm.

23. The cryogel of claim 22, wherein the cryogel is formed according to the process as claimed in claim 1.

24. The cryogel as claimed in claim 21, comprising pores on its surface.

25. The cryogel as claimed in claim 21, comprising less than 10 weight % degradation products of the polysaccharide.

26. The cryogel of claim 21 having a purity of at least 90%.

27. The cryogel as claimed in claim 21 for use as a medicament.

28. The cryogel as claimed in claim 21 for use in tissue augmentation, cosmetic surgery, wound dressing, post surgical adhesion prevention, regenerative medicine applications, tissue engineering applications or for use as a scaffold for the incorporation of cells for tissue repair.

29. The use of the cryogel as claimed in claim 21 for use in the manufacture of a medicament for tissue augmentation, cosmetic surgery, wound dressing, post surgical adhesion prevention, regenerative medicine applications, tissue engineering applications or for use as a scaffold for the incorporation of cells for tissue repair.

30. A method of medical treatment comprising the steps of administering the cryogel as claimed in claim 21 to a patient in need thereof for use in tissue augmentation, cosmetic surgery, wound dressing, post surgical adhesion prevention, regenerative medicine applications, tissue engineering applications or for use as a scaffold for the incorporation of cells for tissue repair.

31. A material for use in tissue augmentation, the material comprising the cryogel as claimed in claim 21.

32. The material as claimed in claim 31, said material being a dermal filler.

33. An injectable particulate composition comprising the cryogel as claimed claim 21.

34. A wound dressing comprising the cryogel as claimed in claim 21.

35. A drug delivery composition comprising the cryogel as claimed in claim 21 and a pharmaceutical or bioactive agent.

36. An anti-aging composition comprising the cryogel as claimed in claim 21.
